(19) United States
(12) Patent Application Publication
(10) Pub. No.: US 2004/0192905 A1
(43) Pub. Date: Sep. 30, 2004

(54) Polymerizable monomers and process of preparation thereof

(76) Inventors: Mohan Gopalkrishna Kulkarni, Maharashtra (IN); Jayant Jagannath Khandare, Maharashtra (IN)

Correspondence Address: NIXON & VANDERHYE, PC, 1100 N GLEBE ROAD, 8TH FLOOR, ARLINGTON, VA, 22201-4714, US

(21) Appl. No.: 10/402,256

(22) Filed: Mar. 31, 2003

(51) Int. Cl. C07H 17/02; C08G 63/48

(52) U.S. Cl. 536/53; 536/119; 525/54.2

(57) ABSTRACT

The present invention relates to polymerizable monomers for applications in medicine and biotechnology and synthesis thereof. The polymerizable ligands containing NAcetyl Glucosamine bind more strongly to lysozyme than NAG itself. The binding is further enhanced when a spacer arm, for example 6-Amino Caproic Acid (6-ACA) is introduced in the structure. The conjugated ligands could be used for prevention and treatment of bacterial and viral infections Moreover these ligands can be coupled to stimuli sensitive polymers and used for the recovery of biomolecules The methodology can be extended to other ligands such as sialic acid and the corresponding polymers used for preventil1g influenza and lor rotavirus infections

### FIELD OF THE INVENTION

This invention relates to polymerizable monomers containing N-Acetyl Glucosamine (NAG) of formula (1) herein below

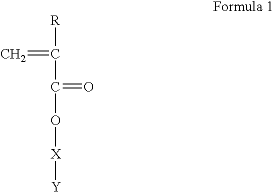

wherein, R is H, CH₃, C₂H₅ or C₆H₅, X is based on 4-Amino Butyric Acid (4-ABA), 6-Amino Caproic Acid (6-ACA), 8 Amino Octanoic Acid (8-AOA), IO-Amino Decanoic Acid (IO-ADA), II-Amino Undecanoic Acid (II-ADA); Y is selected from the group consisting of N-Acetyl Glucosamine, mannose, galactose and sialic acid, fructose, ribulose, erythrolose, xylulose, psicose, sorbose, tagatose, glucopyranose, fructofuninose, deoxyribose, galactosamine, sucrose, lactose, isomaltose, maltose, cellobiose, cellulose and amylose.

More particularly the present invention relates to the said monomers containing carbohydrate ligands and preparation thereof through the specific linkage mentioned herein. Still more particularly it relates to monomers which bind more strongly to lysozyme than NAG itself. The monomers provided are prepared by reacting acryloyl chloride of formula 2 (wherein R=H for Acryloyl and R=CH₃ in case Methacryloyl Chloride) herein below with a ligand such as N-Acetyl Glucosamine of formula 3 exemplified hereinbelow or mannose, galactose and sialic acid.

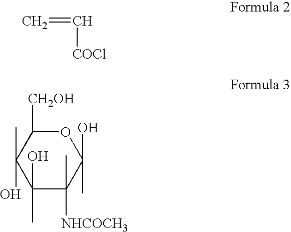

### BACKGROUND OF THE INVENTION

Protein carbohydrate interactions play an important role in biological processes such as cell adhesion and cell recognition e.g. influenza infection is caused by the binding of the virus with the Red-, Blood Cells (RBC) which contain sialic acid. Polymeric ligands that bind to the virus more powerfully than RBC's will prevent the influenza infection (Sigal et al. J. Am. Chem. Society, 118, 16:3789-3800, 1996). Similar binding is also involved in rotavirus infections.

Protein carbohydrate interactions are of low affinity. If relative density and spatial arrangement of ligands incorporated is optimized, then the binding can be substantially enhanced. The enhanced interaction between monomeric ligand with a specific binding site of biomolecule can also find applications in affinity separations, drug delivery and biotechnology. To imitate and exploit this mechanism there is a need to devise simple methodologies for the synthesis of the polymerizable ligands, which will enhance substrate ligand interactions.

Site-specific ligand conjugates are interactive molecules, and are useful in immunoassays and biomolecule separations. The interacting molecules can be proteins or peptides, antibodies, enzymes, polysaccharides or glycoproteins that specifically bind to other substrate receptors in the suitable environment. A ligand so bound can be displaced from the binding site by altering environmental conditions.

Sharon et al., (Science 246:227-234,1989) reported that carbohydrate portions of glyco-conjugate molecules were an important entity in carbohydrate biology. Advantage of carbohydrate modification lies in that it may impart change in physical characteristics such as solubility, stability, activity, antibody recognition and susceptibility to enzymes

Damschroder et al. (U.S. Pat. No. 2,548,520, 1951) disclosed high molecular weight materials prepared by copolymerizing proteins conjugated with unsaturated monomers or proteins conjugated with preformed polymers. Synthesis of these high molecular weight materials generally requires temperatures up to 100 0 C. Such high temperatures are not well tolerated by most of the proteins. Thus the methods described are unsuitable for producing polymers of biologically active molecules.

Jaworek, et al. (U.S. Pat. No. 3,969,287, 1976) reports a method for the preparation of carrier-bound proteins, wherein the protein is reacted with a monomer containing at least one double bond capable of copolymerization The carrier is provided as a water insoluble solid or is produced in situ by the polymerization of water-soluble monomers in the presence of the protein monomer conjugate. The proteins utilized in the method of this invention are typically enzymes

Alternatively, the protein may be conjugated with a polymer to form a polymer protein conjugate. The extent of conjugation of proteins in this is limited by the steric considerations. Moreover the conjugation of the ligand along the polymer chain cannot be precisely arranged, controlled/reproduced.

Monomers and oligomers can be covalently bonded directly to selected ligands through chemical spacer arm to form monomer, oligomer conjugates. This can be followed by the copolymerization of these conjugates with other monomers. Using controlled chemical synthesis methods it is possible to control the spacing, steric accessibility, number of ligand molecules in the polymer molecular weight, density, solubility and physical structure of the polymeric conjugates. The method thus provides unique advantages in various applications.

It is therefore advantageous to synthesize monomers, which are covalently linked to ligands for enhanced binding with substrates. The efficiency of ligand binding with the specific substrates receptors can be quantified in various terms such as binding constants (Kb), association constants (Ka) or the relative inhibition (I 50) in presence of the substrates.

Recent advancements in the field of glycoscience have demonstrated enhanced binding between carbohydrate ligands and specific receptors as a result of the cluster effect These interactions result from intrinsic properties of these ligands. Horejsi et al. (Biochim. Biophys. Acta, 538, 293, 1978) reported synthetic glycoconjugate compounds comprising neoglycolipids, neoglycoproteins and glycopolymers. In order to enhance binding of the carbohydrate ligands in bio and immunochemical assays we have conjugated the ligand N-Acetyl Glucosamine with a monomer containing spacer, which further can be polymerized to a suitable molecular weight.

It is well known that influenza virus hemagglutinin mediates the initial step of infection. This involves binding between the hemagglutinin and the sialic acid residues on the cell surface receptors on the nasal epithelial cells.

Spevak et al. (J. Am. Chem. Society, 115,1146-1147, 1993) reported the polymerized liposomes containing C-glycosides of sialic acid which were potent inhibitors of influenz-a virus. Moreover the authors demonstrated that the infection was inhibited more effectively when the ligand bearing monomer was polymerized. Wu et al., (Biorg. Med. Chem. Lettl 0,341-343,2000) demonstrated the effectiveness of polyacrylamides containing sialic acid groups in inhibiting the attachment of influenza virus to red blood cells

The carbohydrates such as NAG serve as ligands for lectins and lysozyme. Roy et al. (J. Chem. Soc. Chem. Comm., 1611-1613, 1992) reported custom designed glycopolymer synthesis by terpolymerizations. The N-acryloyl precursors and the acrylamide were used as effector molecules to provide specific properties such as hydrophobicity and mimicking tyrosine residues of proteins.

Mammen, et al., (J. Med. Chem., 38, 4179-4190, 1995)reported polyacrylamides bearing pendent alpha sialoside groups as efficient inhibitors in agglutination of erythrocytes by influenza virus, suggesting the role of polyvalency. The affinity of the polyvalent inhibitor towards a surface of the virus is greatly enhanced compared to a monovalent sialic acid inhibitor. In addition high molecular weight polymers containing ligands inhibit binding between the virus and its receptor through steric exclusion Sigel et al. (J. Am. Chem. Society, 118(16),3789-3800, 1996)reported the efficacy of polymers containing sialoside groups in inhibiting the adhesion of influenza virus to erythrocytes. They delineated the contributions of enhanced substrate ligand binding and stene considerations to efficiency of inhibition These investigators have investigated various types of ligands, which can be exploited for the inhibition of the influenza virus Mochalova et al. (Antiviral Research, 23, 179-190, 1994)synthesized glycylamido benzylsialoside with poly(acrylic acid-co-acrylamides)and dextrans. These polymeric ligands were evaluated for their ability to bind to influenza A and B virus strains in cell culture.

Methods of synthesis of oligomers and polymers reported in the literature are complicated and are focussed mainly on the prevention of the viral infection like influenza (Sigal et al., J. Am. Chem. Society, 118, 16, 3789-3800, 1996).

It is reported that the polymeric fucosides are resistant to an enzyme neuraminidase found on the surface of influenza virus. The viruses also cleave sialic acid groups on the Red Blood Cell surfaces from molecules that bind to the surface of the virus, and thereby destroy the cell stability.

Recent literature highlights the advantages of polyvalent interactions and their application in medicine and biotechnology. The fucoside sialic acid moieties can be linked to polymer for the treatment of rotavirus (Mandeville, III, et al., U.S. Pat. No. 6,187,762, 2001). These moieties can inhibit or prevent rotavirus infection in mammals and humans.

Akai, et al. (J. Carbohydrate, Chem., 20(2), 121-143,2001)synthesized styryl monomers containing D-mannopyranose, 2-acetamido-2-deoxy-b-D-mannopyranose, 2-deoxy-2 fluoro-b-D-mannopyranose, and 2-deoxy-b-D-arabino-hexopyranose on their side chains. These were potent inhibitors for exo-a-mannosidase digestion. El-Saied et al. (J ,Carbohydrate,Chem, 18(5),585-602, 1999) synthesized graft copolymers containing polysaccharide backbones and acryloylcyanoaceto hydrazide (ACAH) as chelating monomer The effect of grafting variables such as time, temperature, monomer concentration and initiator onto chitin was studied. Dimick et al. (J. Am. Chem. Society, 121, 44, 10286-10296, 1999) reported the molecular cluster glycoside effects and the synthesis of polyvalent ligands for the plant lectin concanavalin A. Krepinsky, et al. (U.S. Pat. No. 6,184,368, 2001) reported the limitations in the productive binding of chitosan to lysozyme and methods for the synthesis of polyvalent carbohydrate molecules by glycosylation of partially protected polysaccharides bearing a single glycosylating agent or a mixture of glycosylating agents.

Chitosan (Formula 4) is a linear, binary heteropolysaccharide and consists of 2-acetoamido-2-deoxy-β-D-glucose (GlcNAc, A-unit) and 2-amino-2-deoxy -β-D glucose (GlcNAc, D-unit). The active site of lysozyme comprises subsites designated A-F. Specific binding of chitosan sequences to lysozyme begins with binding of the NAG units in the subsite C. Moreover natural ligands derived from glucose are susceptible to microbial growth Hence there is a need to synthesize ligands similar to repeat units of chitosan which will not be hydrolyzed by lysozyme These polymers are expected to be more stable than chitin and chitosan reported earlier

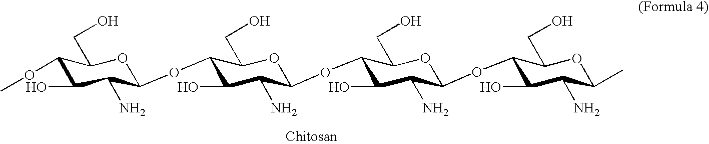

Apart from the type of the ligand, its distribution along the polymer chain also plays a crucial role in influencing the efficiency of the inhibition. Controlled synthesis of amphiphilic block copolymers bearing pendent N-Acetyl-D-Glucosamine residues by living cationic polymerization and the interaction of the resulting diblock copolymers with lectins was reported by Yamada et al. (Macromolecules, 32,3553-3558, 1999) This methodology of synthesizing homopolymers and the block copolymers containing N-Acetyl-D-Glucosamine residues demonstrates significant increase in binding affinity for lectin. Applicability of the method is however limited by need for very low temperature and stringent polymerization conditions.

### OBJECTS OF THE INVENTION

The main object of the present invention is to provide polymerizable monomers for applications in medicine and biotechnology.

Another object is to provide a convenient method of preparation of reactive polymers of various molecular weights with the ligands like NAG, mannose, galactose or stalic acid, fructose, ribulose, erythro lose, xylulose, psicose, sorbose, tagatose, glucopyranose, fructofuranose, deoxyribose, galactosamine, sucrose, lactose, isomaltose, maltose, cellabiose, cellulose and amylose.

Still another object is to provide a convenient method of preparation of monomers, containing a ligand.

Yet another object is to provide a method of preparation of monomers containing NAG for enhanced interactions.

Still another object is to provide more stable ligands for the interactions with biomolecules than the natural polymers such as chitin and chitosan containing NAG.

### SUMMARY OF THE INVENTION

The present invention provides polymerizable monomers for a biomolecular target and method for synthesis thereof, which exhibits selective binding to the target enzyme/protein. The present invention also provides a method for obtaining affinity ligand useful for isolating target biomolecule from a solution. The polymerizable ligands may be further oligomerized or polymerized and may posses a terminal functional group. Further, these can be copolymerized with other comonomers to offer copolymers bearing a wide range of polymer architecture than those realized in the past. These ligands containing N-Acetyl Glucosamine are easy to prepare and are resistant to degradation are reusable, stable and free from microbial contamination,

Accordingly the present invention provides a polymerizable monomer of formula 1

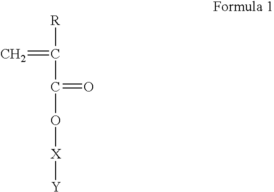

wherein, R is H, CH₃, C₂H₅, C₆H₅, X is a based on spacer exemplified by 4-Amino Butyric Acid (4-ABA), 6-Amino Caproic Acid (6-ACA), 8-Amino Octanoic Acid (8-AOA), IO-Amino Decanoic Acid (10-ADA), II-Amino Undecanoic Acid (11-ADA); Y is a carbohydrate ligand selected from the group consisting of N-Acetyl Glucosamine, mannose, galactose and sialicacid, fructose, ribulose, erythrolose, xylulose, psicose, sorbose, tagatose, glucopyranose, fructofuranose, deoxyribose, galactosamine, sucrose, lactose, isomaltose, maltose, cellobiose, cellulose and amylose.

The present invention also provides a process for the preparation of the polymerizable monomer of formula 1

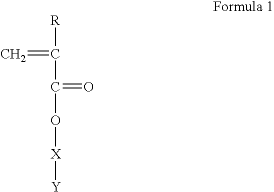

wherein, R is H, CH₃, C₂H₅, C₆H₅, X is a based on spacer exemplified by 4-Amino Butyric Acid (4-ABA), 6-Amino Caproic Acid (6-ACA), 8-Amino Octanoic Acid (8-AOA), IO-Amino Decanoic Acid (10-ADA), II-Amino Undecanoic Acid (11-ADA), Y is a carbohydrate ligand selected from the group consisting of N-Acetyl Glucosamine, mannose, galactose and sialic acid, fructose, ribulose, erythrolose, xylulose, psicose, sorbose, tagatose, glucopyranose, fructofuranose, deoxyribose, galactosamine, sucrose, lactose, isomaltose, maltose, cellobiose, cellulose and amylose, which comprises dissolving a polymerizable monomeric acid chloride in a solution of an alkali, separately preparing an aqueous solution of a spacer, bringing the temperature of the solutions to 5 to 10° C., adding drop wise the solution of polymerizable monomeric acid chloride to the solution of the spacer, maintaining pH of the mixture 7 4 to 7.8 by the addition of the alkali solution, and the temperature 5 to 10° C. during addition removing the unreacted monomeric acid chloride by solvent extraction, acidifying the reaction mixture to pH 5 to 5.5, and solvent extracting the reaction mixture, precipitating using a non solvent to obtain the monomeric-spacer conjugate, drying under vacuum at room temperature, dissolving the conjugate in an organic solvent, adding to this a carbohydrate ligand, adding to this reaction mixture a coupling agent, allowing the reaction for a period of 24 to 48 hrs at room temperature, removing the unreacted coupling agent, treating the clear solution with a solvent to obtain the polymerizable monomer In one embodiment of the invention the polymerizable monomeric acid chloride is selected from methacryloyl or acryloyl chloride.

In another embodiment of the invention, the alkali comprises a 10 to 20% solution of hydroxide, bicarbonate or carbonate of alkali metal exemplified by NaOH, KOH, NaHCO₃, Na₂CO₃.

In another embodiment of the invention, the spacer includes bifunctional compounds having a reactive site for bonding with the monomeric acid chloride and a reactive site for bonding with carbohydrate ligand, functional groups exemplified by OH, COOH or NH₂ such as 4-Amino Butyric (4-ABA)Acid, 6-Amino Caproic Acid (6-ACA), 10-Amino Decanoic Acid (10-ADA), 1,4-diaminobutane, hexamethylenediamine and 1,4-butanediol.

In another embodiment of the invention the solvent used for solvent extraction of unreacted monomeric spacer is non solvent to the monomeric spacer exemplified by ethyl or methyl acetate

In yet another embodiment the acidification is carried out using mineral acids having concentration of 5 to 20%.

In another embodiment the organic solvent used to dissolve the conjugate is selected from dimethyl formamide, tetra hydro furan and di-methyl sulfoxide.

In another embodiment the carbohydrate ligand is selected from NAG, sialic acid, mannose and galactose.

In a further embodiment the coupling agent used is selected from Dicyclohexyl Carbodiimide (DCC), I-Cyclohexyl 3-(2-Morpholinoethyl) Carbodiimide metho-p-toluenesulfonate (CMC), and I-Ethyl-3-(3-Dimethylamino-propyl) Carbodiimide (EDC)

In another embodiment the non-solvent used to precipitate the polymerizable monomer is selected from acetone, diethyl ether and hexane.

In yet another embodiment the molar ratio of monomeric acid chloride to amino acid used for the synthesis of the monomer is 1:1.

In yet another embodiment the molar ratio of coupling agent for condensation of monomeric spacer to carbohydrate ligand is 1:1

In yet another embodiment the molar ratios of polymerizable monomeric acid chloride to spacer is in the range from 0 1:1 to 1:0 1, preferably 0.5 to 1 to 1:0.5, more preferably from 0.8:1 to 1.0:8

In a feature of the present invention the conjugation of the monomer with the ligand is effected through a spacer. The “spacer ” provides greater accessibility to the ligand conjugate for binding with receptor biomolecule.

In yet another feature the polymerizable acid chloride is linked to NAG through CH₂OH group, a feature not present in chitosan, chitin and/or other derivatives of NAG so far reported in the literature

### DETAILED DESCRIPTION OF THE INVENTION

NAG is derived from chitosan which is a linear, binary heteropolysaccharide and consists 2-acetoamido-2-deoxy-β-D-glucose (GlcNAc, A-unit) and 2-amino 2-deoxy-β-D-glucose (GlcNAc, D-unit). Chitosan is a powerful natural ligand, which binds to lysozyme through the NAG residues. But it suffers from three major limitations 1) Chitosan is insoluble at neutral pH, which limits many applications 2) Chitosan undergoes the transglycosylation and mutarotation, which substantially reduces its activity and efficiency 3) Chitosan is hydrolyzed by lysozyme. The present invention provides a simple process for preparation of polymerizable monomers comprising NAG, which can be exploited for multivalent interactions. The merits of the approach have been highlighted using NAG as an illustration. Various methods have been reported in the past for the synthesis of glycoconjugate oligomers and clusters for the receptor binding activity. Nishimora et al. (Macromolecules, 27, 4876-4880, 1994) synthesized clustering sugar homopolymers from acrylamidoalkyl glycosides of N-Acetyl-D-Glucosamine. On addition of the cluster type polymer, binding to WGA was enhanced. The methodology described by us is useful to synthesize the polyvalent carbohydrate conjugates to enhance ligand substrate interactions. Further the approach can be extended to other ligands such as sialic acid, mannose and galactose

The present invention provides polymerizable monomers having formulae (1)

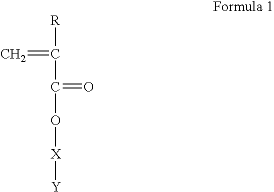

wherein, R is H, CH₃, C₂H₅, C₆H₅; X is a based on spacer exemplified by 4-Amino Butyric Acid (4-ABA), 6-Amino Caproic Acid (6-ACA), 8-Amino Octanoic Acid (8-AOA), IO-Amino Decanoic Acid (10-ADA), II-Amino Undecanoic Acid (11-ADA); Y is a carbohydrate ligand such as N-Acetyl Glucosamine, mannose, galactose and sialicacid, fructose, ribulose, erythrolose, xylulose, psicose, sorbose, tagatose, glucopyranose, fructofuranose, deoxyribose, galactosamine, sucrose, lactose, isomaltose, maltose, cellobiose, cellulose and amylose.

The present invention also provides a process for the preparation of the polymerizable monomers mentioned above which comprises dissolving a polymerizable monomeric acid chloride in a solution of an alkali, separately preparing an aqueous solution of a spacer, bringing the temperature of the solutions to 5 to 10° C., adding drop wise the solution of polymerizable monomeric acid chloride to the solution of the spacer, maintaining pH of the mixture 7.4 to 7.8 by the addition of the alkali solution, and the temperature 5 to 10° C. during addition removing the unreacted monomeric acid chloride by solvent extraction, acidifying the reaction mixture to pH 5 to 5 5, and solvent extracting the reaction mixture, precipitating using a non solvent to obtain the monomeric-spacer conjugate, drying under vacuum at room temperature, dissolving the conjugate in an organic solvent, adding to this a carbohydrate ligand, adding to this reaction mixture a coupling agent, allowing the reaction for a period of 24 to 48 hrs at room temperature, removing the unreacted coupling agent, treating the clear solution with a solvent to obtain the polymerizable monomer.

The polymerizable monomeric acid chloride is preferably selected from methacryloyl or acryloyl chloride. In another embodiment the alkali comprises 10 to 20% solution of hydroxide, bicarbonate or carbonate of alkali metal exemplified by NaOH, KOH, NaHCO₃, Na₂CO₃. The spacer may include bifunctional compounds having a reactive site for bonding with the monomeric acid chloride and a reactive site for bonding with carbohydrate ligand, functional groups exemplified by OH, COOH or NH₂ such as 4-Amino Butyric (4-ABA)Acid ,6-Amino Caproic Acid (6-ACA), 10-Amino Decanoic Acid (10-ADA), 1,4-diaminobutane, hexamethylenediamine, 1,4-butanediol. The solvent used for solvent extraction of unreacted monomeric spacer may be non solvent to the monomeric spacer exemplified by ethyl or methyl acetate. The acidification may be done by using mineral acids having concentration of 5 to 20%. The organic solvent used to dissolve the conjugate may be such as dimethyl formamide, tetra hydro furan or di-methyl sulfoxide The carbohydrate ligand is NAG, sialic acid, mannose or galactose. The coupling agent used is selected from compounds such as Di Cyclohexyl Carbodiimide (DCC), 1-Cyclohexyl 3-(2-Morpholinoethyl) Carbodiimide metho-p-toluenesulfonate (CMC), I-Ethyl-3-(3-Dimethylamino-propyl) Carbodiimide (EDC).

The non solvent used to precipitate the polymerizable monomer is selected from acetone, diethyl ether or hexane. The molar ratio of monomeric acid chloride to amino acid used for the synthesis of the monomer is 1:1 The molar ratio of coupling agent for condensation of monomeric spacer to carbohydrate ligand is 1.1. The molar ratios of polymerizable monomeric acid chloride to spacer are in the range from 0.1:1 to 1:01, preferably 0.5 to 1 to 1:0.5, more preferably from 0.8:1 to 1:08

In a feature of the present invention the conjugation of the monomer with the ligand is preferably effected through a spacer. The “spacer ” provides greater accessibility to the ligand conjugate for binding with receptor biomolecule.

In yet another feature the polymerizable acid chloride is linked to NAG through CH₂OH group, a feature not present in chitosan, chitin and/or other derivatives of NAG so far reported in the literature. In yet another feature the method used for estimation of the relative inhibition is in terms of 1₅₀ mM and Iₘₐₓ mM values, In yet another feature the binding between lysozyme and the monomeric ligand-containing NAG is enhanced

The process reported herein for the incorporation of NAG into monomers is relatively simple Besides the monomers are effective at very low ligand concentration, which is an advantage when the ligands under consideration are expensive e g sialic acid.

It is also expected that the presence of multiple NAG ligands in the polymer backbone will enhance binding to the viruses and biomolecules such as influenza, rotavirus, Wheat Germ Agglutinin (WGA). The polymers containing multiple ligands can potentially interact with multiple receptors simultaneously thereby enhancing the binding to lysozyme. The ability of these ligands to inhibit enzyme activity provides new ways of developing effective inhibitors. The monomers synthesized indicate enhanced substrate ligand interactions and can be used in diverse applications such as in immunoassays and affinity separations. The present invention relates to the monomers containing carbohydrate moieties and preparation thereof The monomer may comprise a spacer arm, which is inserted between the vinyl group and the ligand These monomers may be used for the synthesis of homopolymers, oligomers and copolymers for the recovery of biomolecules.

The polymers comprising carbohydrate monomer conjugates can also further be used in the treatment of bacterial or viral infections, and are expected not to cause drug resistance.

Monomers containing NAG exhibit enhanced hydrolytic stability and water solubility than natural polymers containing NAG such as chitosan . The monomers containing NAG may be used for polymerization or oligomerization. They may be also used as anti infective agents both for prevention and treatment of diseases, recovery of the naturally occurring as well as genetically manipulated biomolecules.

Site-specific interactions in general and protein-carbohydrate interactions in particular are key to enhanced binding. The monovalent interactions are weak whereas multivalent interactions can lead to effective inhibition even at very low concentration. The present invention relates to the polymerizable monomers containing NAG which can be converted to homo and copolymers for applications in medicine and biotechnology. A further aspect of the invention is to prepare monomeric NAG comprising a spacer arm. The advantage of incorporating spacer arms is enhanced accessibility of the ligand to active site of the enzyme.

The term “monomer” means any polymerizable organic compound, which is capable of forming covalent linkages i.e., polymerization under the appropriate conditions can be used such as acrylic or methacrylic acid, acryloyl or methacryloyl chloride, glycidyl acrylate or methacrylate, glycerol acrylate or methacrylate, allyl chloride, hydroxy-lower-alky-1-acrylates, such as 2-hydroxyethyl methacrylate or 3-hydroxypropyl methacrylate, and amino-lower-alkyl acrylates, such as 2-amino-ethyl methacrylate Monomers, which are soluble in water or water/polar organic solvent mixtures, are particularly preferred.

A representative ligand used here is Methacryloyl N-Acetyl Glucosamine of formula 5 as shown herein below but does not limit the scope of invention.

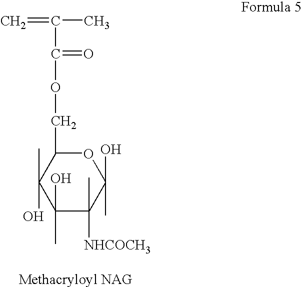

The approach described herein is a generic one and can be extended to other systems as well. For example sialic acid ligands are known to bind to influenza virus and rotavirus. Hence polymers comprising sialic acid can be expected to bind to the two viruses more strongly than the corresponding monomers.

The present invention provides methods for the preparation of polymerizable monomers containing N-Acetyl Glucosamine, which can be oligomerized or polymerized as desired These monomers provide improved binding and inhibition of biomolecules and their efficacy can be further enhanced by polymerization. The polymerizable monomers provided by the present invention may comprise a spacer arm, which is inserted between the vinyl group and the carbohydrate ligand. These monomers are useful for the synthesis of homopolymers, oligomers and copolymers for inhibition of viral infections and the recoveries of biomolecular.

The process for the preparation of the polymerizable ligands is illustrated herein below with reference to examples which are illustrative only and should not be construed to limit the scope of the present invention in any manner.

### EXAMPLE 1

Preparation of Acryloyl N-Acetyl Glacosamine (Ac. NAG)

11. 1 gm. N-Acetyl Glucosamine and 4.2 gm of sodium bicarbonate was dissolved in a beaker, which was equipped with a dropping funnel and a pH meter. The clear solution was stirred continuously on a magnetic stirrer at 5° C. 5 ml Acryloyl Chloride in 5 ml of dichloromethane was added drop wise.

The reaction mixture was maintained at pH 7.5 with addition of saturated solution of sodium bicarbonate. After addition of Acryloyl Chloride, unreacted Acryloyl Chloride was extracted in 100-ml ethyl acetate The clear aqueous solution was separated and acidified to pH 5.0 by the addition of concentrated HCl. Finally Acryloyl N-Acetyl Glucosamine was precipitated in distilled acetone The product was reprecipitated in acetone.

### EXAMPLE 2

Preparation of Methacryloyl 6-Amino Caproic Acid (M Ac. 6-ACA)

250 ml capacity beaker was equipped with dropping funnel and pH meter. 13.16 gm 6ACA, 4 gm. sodium hydroxide and 80 ml. water was stirred continuously at 5 0 C on a magnetic stirrer. Nine milliliter of Methacryloyl Chloride in 10 ml dichlorQmeJhane was added drop wise to the above solution The pH of reaction mixture was maintained at 7.5 by the addition of 10M NaOH solution. Unreacted acid chloride was extracted in 100 ml ethyl acetate The clear aqueous solution was acidified to pH 5.0 using concentrated HCl and the product was extracted in ethyl acetate (3×100 ml). The organic layer was dried on anhydrous sodium sulfate and concentrated under vacuum. The viscous liquid was added to 500 ml petroleum ether. The solid product was obtained and vacuum dried for 48 hrs.

### EXAMPLE 3

Preparation of Acryloyl 6-Amino Caproic Acid N-Acetyl Glucosamine (Ac. 6ACA NAG)

5 gm of Acryloyl 6-Amino Caproic Acid (Ac. 6 ACA) and 5 97 gm. N-Acetyl lucosamine was dissolved in 20 ml dry Di Methyl Formamide (DMF). Clear solution was obtained by continuous stirring and 5.57 gm of Di Cyclohexyl Carbodiimide (DCC) as the coupling reagent was added. The reaction mixture was stirred continuously for 24 hrs. at room temperature. Di Cyclohexyl Urea (DCU) was filtered off and the monomer containing spacer and ligand NAG was precipitated in distilled acetone. It was vacuum dried for 48 hrs.

### EXAMPLE 4

Estimation of binding constant (K_{b}) of monomers containing NAG by fluorescence spectrophotometric method and the enhancement resulting from conjugation with monomers and monomer containing spacer.

Fluorescence spectra of lysozyme were recorded on a Perkin Elmer LS-50 B luminescence spectrophotometer. Excitation frequency was 285 nm, Solutions of lysozyme and N-Acetyl Glucosamine were prepared in 0.066 M phosphate buffer pH 6.2, containing 0.0154 M sodium chloride and 0.008 M sodium azide. 0 1 milliliter of lysozyme 80 μg/ml was mixed with solution containing different ligand concentration in a 2 ml capacity 10 mm square quartz cells maintained at 18 0 C.

Phosphate buffer was added to make the volume to 2 ml. The fluorescence intensities of the solutions were measured, relative to the solutions containing enzymes and buffer mixtures of the identical concentrations reference. The relative fluorescence intensity of lysozyme saturated with solution containing different ligand concentration, Foe, was extrapolated from the experimental values by plotting 1/(F₀-F) against 1/[S] where F is the measured fluorescence of a solution containing enzyme with given substrate concentration [S] and Fo is the fluorescence of a solution of enzyme alone (Chipman et al., J. Biol. Chem., 242-19, 4388-4394, 1967), The highest concentration of polymer substrates was used when enzyme was saturated more than 85%.

TABLE 1
Binding Constants for Monomers Containing NAG

| | Mol. Wt. | Kb |
|---|---|---|
| NAG | 221 | 5.24 × 10 J. |
| Ac. NAG | 275 | 7.04 × 104 |
| Ac. 6A CA.NAG | 404 | 1.97 × 10 |

The binding constants of polymerizable monomers are summarized in Table 1 wherein, N-Acetyl Glucosamine has binding constant 5.24×102 where as that for the monomer Ac. NAG is 7.07'104. The increase in binding constant is 74 times.

With the incorporation of spacer arm 6-ACA the binding constants is further increased to 1.97×10 5, almost 2650 times compared to N-Acetyl Glucosamine.

### EXAMPLE 5

This example describes the estimation of inhibition of lysozyme by monomers Micrococcus lysodeikticus is a substrate for the enzyme lysozyme. Relative binding of monomers and monomers linked to NAG through the spacer arm was estimated by using a procedure reported by Neuberger and Wilson (1967).

1 5% w/v stock solutions of monomeric ligands was prepared in 0.0066 M phosphate buffer pH 6.2 containing 0.01 54 m sodium chloride and 0.008 M sodium azide. One milliliter of stock solution containing different ligand concentration was mixed with 1.6 ml of 78 Jig/ml of Micrococcus lysodeikticus in a 3-ml capacity glass cuvette. The mixture was incubated for 5 minutes at 20 0 C. To this mixture 0.1 ml of lysozyme (27 μg/ml) was added and mixed thoroughly. The relative absorbance at 450 nm (Δ₄₅₀) was recorded for 30 seconds. A blank reading without the ligand was noted and the change in the absorbance per second was calculated. Then relative inhibition was calculated

TABLE 2
Estimation of Relative Inhibition of Lysozyme by Monomers
Containing NAG

| | Mol. Wt. | I₅₀ mM | Iₘₐₓ | 1ₘₐₓ mM |
|---|---|---|---|---|
| NAG | 221 | 74.00 | 55.29 | 92.5 |
| Ac. NAG | 275 | 14.81 | 50.00 | 14.81 |
| Ac 6ACA.NAG | 404 | 0.035 | 52.5 | 0.036 |

The relative inhibition of lysozyme in terms of I 50 for monomer NAG is 74.00 mM and has decreased to 14 81 mM which is almost 5 times lower. Whereas the Iₘₐₓ has decreased from 55.29 to 50 for the monomer containing NAG.

I max has decreased from 55 29 mM to 14.81 mM (Table 2).

The relative inhibition I 50 has decreased from 74 mM to 0.035 mM which is almost to 2110 folds lower than that for NAG indicating enhanced efficacy of inhibition.

We claim:

1. A polymerizable monomer of formula 1 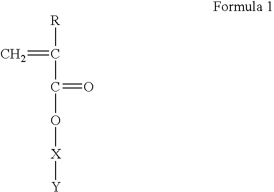
wherein, R is H, CH₃, C₂H₅, C₆H₅; X is a based on spacer exemplified by 4-Amino Butyric Acid (4-ABA), 6-Amino Caproic Acid (6-ACA), 8-Amino Octanoic Acid (8-AOA), IO-Amino Decanoic Acid (10-ADA), 11-Amino Undecanoic Acid (11-ADA)) Y is a carbohydrate ligand selected from the group consisting of N-Acetyl Glucosamine, mannose, galactose and sialicacid, fructose, ribulose, erythrolose, xylulose, psicose, sorbose, tagatose, glucopyranose, fructofuranose, deoxyribose, galactosamine, sucrose, lactose, isomaltose, maltose, cellobiose, cellulose and amylose.

2. A process process for the preparation of the polymerizable monomer of formula 1 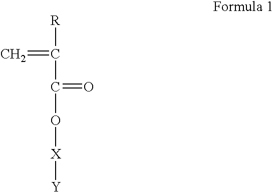
wherein, R is H, CH₃, C₂H₅, C₆H₅; X is a based on spacer exemplified by 4-Amino Butyric Acid (4-ABA), 6-Amino Caproic Acid (6-ACA), 8-Amino Octanoic Acid (8-AOA), IO-Amino Decanoic Acid (10-ADA), II-Amino Undecanoic Acid (11-ADA); Y is a carbohydrate ligand selected from the group consisting of N-Acetyl Glucosamine, mannose, galactose and sialicacid, fructose, ribulose, erythrolose, xylulose, psicose, sorbose, tagatose, glucopyranose, fructofuranose, deoxyribose, galactosamine, sucrose, lactose, isomaltose, maltose, cellobiose, cellulose and amylose, which comprises dissolving a polymerizable monomeric acid chloride in a solution of an alkali, separately preparing an aqueous solution of a spacer, adding drop wise the solution of polymerizable monomeric acid chloride to the solution of the spacer to obtain a mixture, removing the unreacted monomeric acid chloride by solvent extraction, acidifying the reaction mixture, and solvent extracting the reaction mixture, precipitating the extract using a non-solvent to obtain a monomeric-spacer conjugates drying the monomeric-spacer conjugate under vacuum and then dissolving the conjugate in an organic solvent, adding to this solution a carbohydrate ligand to obtain a reaction mixture, adding a coupling agent to the reaction mixture, allowing the reaction to occur and then removing the unreacted coupling agent, treating the clear solution with a solvent to obtain the polymerizable monomer.

3. A process as claimed in claim 2 wherein the temperature of the bringing the temperature of the polymerizable monomeric acid chloride solution in alkali and aqueous solution of a spacer solution are brought to a temperature in the range of 5 to 10° C. before the polymerizable monomeric acid chloride solution is added to the spacer solution.

4. A process as claimed in claim 2 wherein the pH of the mixture is maintained in the range of 7.4 to 7.8 by addition of an alkali solution and the temperature is maintained in the range of 5 to 10° C. during addition.

5. A process as claimed in claim 2 wherein the reaction mixture of polymerizable monomeric acid chloride in alkali solution and the aqueous spacer solution is acidified to a pH in the rang of 5to 5.5.

6. A process as claimed in claim 2 wherein the reaction using the coupling agent is carried out for a period in the range of 24 to 48 hrs and at room temperature.

7. A process as claimed in claim 2 wherein the polymerizable monomeric acid chloride is selected from methacryloyl and acryloyl chloride,

8. A process as claimed in claim 2 wherein the alkali is a 10 to 20% solution of hydroxide, bi-carbonate or carbonate of alkali metal.

9. A process as claimed in claim 8 wherein the alkali is selected from the group consisting of NaOH, KOH, NaHCO₃, and Na₂CO₃.

10. A process as claimed in claim 2 wherein the spacer comprises a bifunctional compound having a reactive site for bonding with the monomeric acid chloride and a reactive site for bonding with carbohydrate ligand, the functional groups being selected from the group consisting of OH, COOH or NH₂ such as 4-Amino Butyric (4-ABA)Acid, ⁶-Amino Caproic Acid (6-ACA), 10-Amino Decanoic Acid (10-ADA), 1,4-diaminobutane, hexamethylenediamine and 1,4-butanediol.

11. A process as claimed in claim 2 wherein the solvent used for solvent extraction of unreacted monomeric spacer is non-solvent to the monomeric spacer and is selected from ethyl acetate and methyl acetate.

12. A process as claimed in claim 2 wherein the acidification is carried out using a mineral acid having concentration of 5 to 20%.

13. A process as claimed in claim 2 wherein the organic solvent used to dissolve the conjugate is selected from dimethyl formamide, tetra hydro furan and di-methyl sulfoxide.

14. A process as claimed in claim 2 wherein the carbohydrate ligand is selected from NAG, sialic acid, mannose and galactose

15. A process as claimed in claim 2 wherein the coupling agent used is selected from compounds such as Dicyclohexyl Carbodiimide (DCC), I-Cyclohexyl 3-(2-Morpholinoethyl) Carbodiimide metho-p-toluenesulfonate (CMC), and I-Ethyl-3-(3-Dimethylamino-propyl) Carbodiimide (EDC).

16. A process as claimed in claim 2 wherein the non solvent used to precipitate the polymerizable monomer is selected from acetone, diethyl ether and hexane.

17. A process as claimed in claim 2 wherein the molar ratio of monomeric acid chloride to amino acid used for the synthesis of the monomer is 1:1,

18. A process as claimed in claim 2 wherein the molar ratio of coupling agent for condensation of monomeric spacer to carbohydrate ligand is 1:1

19. A process as claimed in claim 2 wherein the molar ratio of polymerizable monomeric acid chloride to spacer is in the range of 0.1 1 to 1 0. 1.

20. A process as claimed in claim 19 wherein the molar ratio of polymerizable monomeric acid chloride to spacer is in the range of 0.5 to 1 to 1:0 5

21. A process as claimed in claim 19 wherein the molar ratio of polymerizable monomeric acid chloride to spacer is in the range of 0. 8 1 to 1:0 8.

22. A process as claimed in claim 2 wherein the polymerizable acid chloride is linked to NAG through CH₂OH group.
